(19) United States
(12) Patent Application Publication
(10) Pub. No.: US 2025/0340586 A1
(43) Pub. Date: Nov. 6, 2025

(54) PREPARATION OF HIGHLY CONCENTRATED MRNA

(71) Applicant: ModernaTX, Inc., Cambridge, MA (US)

(72) Inventors: Mark Geng, Cambridge, MA (US); James Sinoimeri, Cambridge, MA (US)

(73) Assignee: ModernaTX, Inc., Cambridge, MA (US)

(21) Appl. No.: 18/866,285

(22) PCT Filed: May 16, 2023
(86) PCT No.: PCT/US2023/067077

§ 371 Date: Nov. 15, 2024

Related U.S. Application Data
(60) Provisional application No. 63/342,995, filed on May 17, 2022.

(51) Int. Cl. C07H 21/02

(52) U.S. Cl. CPC C07H 21/02

(57) ABSTRACT

Provided herein are compositions of highly concentrated mRNA and related methods for preparation and use of the compositions as mRNA process intermediates in the synthesis of therapeutic and prophylactic mRNA formulations.

### RELATED APPLICATION

This application claims the benefit under 35 U.S.C. § 119 (e) to U.S. Provisional Application No. 63/342,995, entitled “PREPARATION OF HIGHLY CONCENTRATED MRNA,” filed on May 17, 2022, the entire contents of which are incorporated herein by reference.

### REFERENCE TO AN ELECTRONIC SEQUENCE LISTING

The contents of the electronic sequence listing (M137870237WO00-SEQ-VLJ.xml; Size: 11,510 bytes; and Date of Creation: May 16, 2023) is herein incorporated by reference in its entirety.

### BACKGROUND

mRNA formulations are prepared using in vitro transcription (IVT) reactions followed by downstream processing events. Current methods of preparing mRNA typically involve performing IVT and downstream processing events in a continuous process in order to avoid rapid degradation associated with mRNA process intermediates, which are unstable during storage. Methods that improve storage of mRNA process intermediates will allow for non-continuous methods of preparing mRNA formulations and higher quality product.

### SUMMARY

Stable mRNA process intermediates, which can be stored for long periods of time with minimal loss in mRNA integrity and purity have been developed. In some aspects, a composition comprising a gel or viscous liquid comprising mRNA in a concentration of at least 10 g/L is provided. In some embodiments the mRNA does not comprise a cap. In some embodiments the composition comprises a diafiltered gel.

In some embodiments the composition is a process intermediate, packaged for storage at refrigerated or colder temperatures. In some embodiments the composition is in a container having a volume capacity of at least one liter.

In some embodiments the mRNA is in a concentration of at least 11 g/L. In some embodiments the mRNA is in a concentration of at least 12 g/L. In some embodiments the mRNA is in a concentration of at least 13 g/L. In some embodiments the mRNA is in a concentration of at least 14 g/L. In some embodiments the mRNA is in a concentration of at least 15 g/L. In some embodiments the mRNA is in a concentration of at least 16 g/L. In some embodiments the mRNA is in a concentration of at least 17 g/L. In some embodiments the mRNA is in a concentration of at least 18 g/L. In some embodiments the mRNA is in a concentration of at least 19 g/L. In some embodiments the mRNA is in a concentration of at least 20 g/L. In some embodiments the mRNA is in a concentration of at least 21 g/L. In some embodiments the mRNA is in a concentration of at least 22 g/L. In some embodiments the mRNA is in a concentration of at least 23 g/L. In some embodiments the mRNA is in a concentration of at least 24 g/L. In some embodiments the mRNA is in a concentration of at least 25 g/L. In some embodiments the mRNA is in a concentration of at least 26 g/L. In some embodiments the mRNA is in a concentration of at least 27 g/L. In some embodiments the mRNA is in a concentration of at least 28 g/L. In some embodiments the mRNA is in a concentration of at least 29 g/L. In some embodiments the mRNA is in a concentration of at least 30 g/L.

In some embodiments the mRNA is in a concentration of less than 30-35 g/L. In some embodiments the mRNA is in a concentration of less than 28 g/L. In some embodiments the mRNA is in a concentration of less than 29 g/L. In some embodiments the mRNA is in a concentration of less than 30 g/L. In some embodiments the mRNA is in a concentration of less than 31 g/L. In some embodiments the mRNA is in a concentration of less than 32 g/L. In some embodiments the mRNA is in a concentration of less than 33 g/L. In some embodiments the mRNA is in a concentration of less than 34 g/L. In some embodiments the mRNA is in a concentration of less than 35 g/L.

In some embodiments the mRNA has enhanced stability at refrigerated temperatures relative to a corresponding mRNA composition in a concentration of 3 g/L-6 g/L. In some embodiments the enhanced stability is measured as tail purity and/or size purity.

A method of preparing an mRNA formulation is provided in some aspects. The method involves diluting an overconcentrated mRNA composition to form a dilute mRNA composition, wherein the overconcentrated mRNA composition comprises a gel or viscous liquid comprising mRNA in a concentration of at least 10 g/L and mixing the dilute mRNA composition with one or more carrier compounds to produce an mRNA formulation.

In some embodiments the overconcentrated mRNA composition is stored at refrigerated temperatures. In some embodiments the overconcentrated mRNA composition is stored at refrigerated temperatures for at least 1 day. In some embodiments the overconcentrated mRNA composition is stored at refrigerated temperatures for 1 to 90 days.

In some embodiments the overconcentrated mRNA composition is concentrated to at least 11 g/L. In some embodiments the overconcentrated mRNA composition is concentrated to at least 12 g/L. In some embodiments the overconcentrated mRNA composition is concentrated to at least 13 g/L. In some embodiments the overconcentrated mRNA composition is concentrated to at least 14 g/L. In some embodiments the overconcentrated mRNA composition is concentrated to at least 15 g/L. In some embodiments the overconcentrated mRNA composition is concentrated to at least 16 g/L. In some embodiments the overconcentrated mRNA composition is concentrated to at least 17 g/L. In some embodiments the overconcentrated mRNA composition is concentrated to at least 18 g/L. In some embodiments the overconcentrated mRNA composition is concentrated to at least 19 g/L. In some embodiments the overconcentrated mRNA composition is concentrated to at least 20 g/L. In some embodiments the overconcentrated mRNA composition is concentrated to at least 21 g/L. In some embodiments the overconcentrated mRNA composition is concentrated to at least 22 g/L. In some embodiments the overconcentrated mRNA composition is concentrated to at least 23 g/L. In some embodiments the overconcentrated mRNA composition is concentrated to at least 24 g/L. In some embodiments the overconcentrated mRNA composition is concentrated to at least 25 g/L. In some embodiments the overconcentrated mRNA composition is concentrated to at least 26 g/L. In some embodiments the overconcentrated mRNA composition is concentrated to at least 27 g/L. In some embodiments the overconcentrated mRNA composition is concentrated to at least 28 g/L. In some embodiments the overconcentrated mRNA composition is concentrated to at least 29 g/L. In some embodiments the overconcentrated mRNA composition is concentrated to at least 30 g/L.

In some embodiments the mRNA is in a concentration of less than 28 g/L, 29 g/L, 30 g/L, 31 g/L, 32 g/L, 33 g/L, 34 g/L, or 35 g/L.

In some embodiments the overconcentrated mRNA composition is produced by an IVT reaction and a concentration step. In some embodiments the IVT reaction is a quantitative IVT (qIVT) reaction.

In some embodiments a purification step is performed following the IVT reaction. In some embodiments the purification step is tangential flow filtration (TFF).

In some embodiments the overconcentrated mRNA composition is a gel. In some embodiments the overconcentrated mRNA composition is a viscous liquid.

In some embodiments the overconcentrated mRNA composition is diafiltered before the dilute mRNA composition is prepared.

In some embodiments the overconcentrated mRNA composition is subjected to a downstream processing step before mixing the dilute mRNA composition with one or more carrier compounds.

In some embodiments the overconcentrated mRNA composition is subjected to a downstream processing step before the dilute mRNA composition is prepared. In some embodiments the downstream processing step is a cap reaction step, and/or a chromatography step.

In some embodiments the overconcentrated mRNA composition is produced and diluted and optionally the downstream processing step is performed in a continuous manufacturing process.

In some embodiments the overconcentrated mRNA composition is produced and diluted and optionally the downstream processing step is performed in a in a non-continuous manufacturing process.

In some embodiments the dilute mRNA composition has a concentration range of 3 g/L to 6 g/L.

In some embodiments the one or more carrier compounds is a lipid. In some embodiments the lipid comprises a lipid nanoparticle (LNP).

### BRIEF DESCRIPTION OF THE DRAWINGS

FIG. 1 is a schematic of the IVT TFF process. Arrows are used to designate the flow of material.

FIG. 2 is a schematic visualization of the experimental summary for analyzing the tail purity data.

FIG. 3 is the percent tail purity of post- and pre-gel overconcentration events.

FIG. 4 is the distribution of the percent tail purity of post- and pre-gel overconcentration events.

FIG. 5 is the variance of the percent tail purity of post- and pre-gel overconcentration events.

FIG. 6A is graph showing the tail purity of highly concentrated mRNA process intermediates (IVT TFF and qIVT process intermediates) stored at different temperatures (4° C. and RT) over a period of 65 days relative to a dilute mRNA process intermediate (FFB).

FIG. 6B is graph showing the size purity of highly concentrated mRNA process intermediates (IVT TFF and qIVT process intermediates) stored at different temperatures (4° C. and RT) over a period of 65 days relative to a dilute mRNA process intermediate (FFB).

FIG. 7 is a schematic of the experimental arms to determine how overconcentration event effect product quality attributes.

### DETAILED DESCRIPTION

mRNA compositions for therapeutic or prophylactic uses are typically formulated in a carrier such as a lipid nanoparticle (LNP). mRNA is prepared, purified and mixed with a LNP and then stored for later administration. The mRNA that is mixed with the LNP is first prepared by an in vitro transcription (IVT) reaction, followed by purification steps. The mRNA in this IVT composition mixture is not usually stored for long periods of time because of the relative instability of the mRNA in the preparation. Prolonged storage typically results in significant degradation of the mRNA, which is then unsuitable for the preparation of a drug product.

mRNA preparations having enhanced stability, and capable of being stored, are disclosed herein. The present disclosure includes compositions of highly concentrated mRNA preparations having enhanced stability, mRNA formulations and drug products made from the highly concentrated mRNA preparations and methods of making and using the preparations and formulations.

In some aspects, the present disclosure provides a composition comprising an overconcentrated mRNA composition or preparation. An overconcentrated mRNA composition or preparation, also referred to herein as a highly concentrated mRNA composition or preparation, is a composition comprising mRNA in a concentration of at least 10 g/L.

In some embodiments, the composition comprising mRNA has a concentration of 20-25, 21-25, 22-25, 23-25, 24-25, 25-30, 26-30, 27-30, 28-30, 29-30 g/L. In some embodiments the overconcentrated mRNA composition is concentrated to at least 11 g/L. In some embodiments the overconcentrated mRNA composition is concentrated to at least 12 g/L. In some embodiments the overconcentrated mRNA composition is concentrated to at least 13 g/L. In some embodiments the overconcentrated mRNA composition is concentrated to at least 14 g/L. In some embodiments the overconcentrated mRNA composition is concentrated to at least 15 g/L. In some embodiments the overconcentrated mRNA composition is concentrated to at least 16 g/L. In some embodiments the overconcentrated mRNA composition is concentrated to at least 17 g/L. In some embodiments the overconcentrated mRNA composition is concentrated to at least 18 g/L. In some embodiments the overconcentrated mRNA composition is concentrated to at least 19 g/L. In some embodiments the overconcentrated mRNA composition is concentrated to at least 20 g/L. In some embodiments the overconcentrated mRNA composition is concentrated to at least 21 g/L. In some embodiments the overconcentrated mRNA composition is concentrated to at least 22 g/L. In some embodiments the overconcentrated mRNA composition is concentrated to at least 23 g/L. In some embodiments the overconcentrated mRNA composition is concentrated to at least 24 g/L. In some embodiments the overconcentrated mRNA composition is concentrated to at least 25 g/L. In some embodiments the overconcentrated mRNA composition is concentrated to at least 26 g/L. In some embodiments the overconcentrated mRNA composition is concentrated to at least 27 g/L. In some embodiments the overconcentrated mRNA composition is concentrated to at least 28 g/L. In some embodiments the overconcentrated mRNA composition is concentrated to at least 29 g/L. In some embodiments the overconcentrated mRNA composition is concentrated to at least 30 g/L.

In some embodiments the mRNA is in a concentration of less than 30-35 g/L. In some embodiments the mRNA is in a concentration of less than 28 g/L. In some embodiments the mRNA is in a concentration of less than 29 g/L. In some embodiments the mRNA is in a concentration of less than 30 g/L. In some embodiments the mRNA is in a concentration of less than 31 g/L. In some embodiments the mRNA is in a concentration of less than 32 g/L. In some embodiments the mRNA is in a concentration of less than 33 g/L. In some embodiments the mRNA is in a concentration of less than 34 g/L. In some embodiments the mRNA is in a concentration of less than 35 g/L.

In some embodiments, the composition is a process intermediate, packaged for storage at refrigerated or colder temperatures. In some embodiments, the composition is a process intermediate, packaged for storage at refrigerated temperatures. In some embodiments, the composition is a process intermediate, packaged for storage at colder temperatures. In some embodiments, the high concentration mRNA formulation is more stable relative to the dilute mRNA formulation at temperatures below room temperature. In some embodiments the temperature is refrigerated temperature. Refrigerated temperature, as used herein, refers to temperatures at or below 5° C. In some embodiments refrigerated temperatures are −10 to 5° C. In some embodiments frozen temperatures are at or below −15° C. A highly concentrated mRNA formulation is more stable than a corresponding dilute mRNA formulation if it has less mRNA degradation when stored under the same conditions, i.e., refrigerated temperatures, for a given period of time. In some embodiments, the relative stability of the high concentration mRNA formulation may be assessed using a tail purity or size purity assay. In some embodiments, the high concentration mRNA formulation has enhanced stability at refrigerated temperatures relative to a corresponding mRNA composition in a concentration of about 3-6 (e.g., 1-6, 1-5, 1-4, 1-3, 1-2, 2-6, 2-5, 2-4, 2-3, 3-6, 3-5, 3-4, 4-6, 4-5, 5-6) g/L. In some embodiments, the corresponding mRNA composition in a concentration of about 1 g/L, 2 g/L, 3 g/L, 4 g/L, 5 g/L, 6 g/L. In some embodiments, the corresponding mRNA composition in a concentration of about 1 g/L. In some embodiments, the corresponding mRNA composition in a concentration of about 2 g/L. In some embodiments, the corresponding mRNA composition in a concentration of about 3 g/L. In some embodiments, the corresponding mRNA composition in a concentration of about 4 g/L. In some embodiments, the corresponding mRNA composition in a concentration of about 5 g/L. In some embodiments, the corresponding mRNA composition in a concentration of about 6 g/L. In some embodiments, the corresponding mRNA composition in a concentration of about 3-6 g/L.

In some embodiments the composition comprises a gel or viscous liquid. A gel is a semi-solid, substantially cross-linked system, which exhibits little if any flow in the steady-state. A viscous liquid is a formulation which exhibits some properties of a gel, however, some flow of molecules is enabled. The high concentrations of mRNA in the composition, lead to the formation of viscous liquid or gel states of the composition. In some embodiments, the composition is a gel or viscous liquid comprising mRNA. In some embodiments, the composition is a gel comprising mRNA. In some embodiments, the composition is a viscous liquid comprising mRNA.

The highly concentrated preparation can be used as an mRNA process intermediate during the preparation process, which enables separation of preparation steps. For example, mRNA formulations may be prepared using in IVT reaction and downstream processing events. The formation of a high concentration mRNA formulation results in an mRNA process intermediate that may be stored for longer periods of time relative to dilute mRNA process intermediates. As a result, the mRNA formulation may be prepared in either continuous steps or non-continuous steps. For instance, a mRNA process intermediate from an IVT reaction may be stored and separated from downstream processing events using this method. The methods may also be used to improve quality of mRNA formulations in storage conditions.

Thus, in some embodiments the composition is a process intermediate, which is further manipulated to produce a formulated drug product. Thus, the highly concentrated mRNA preparation may be produced from an IVT reaction product in an unpurified form, or it may be subjected to purification steps or it may be further processed into a pure mRNA product that is ready for drug product formulation. In each instance, the highly concentrated material is a process intermediate.

In some embodiments, highly concentrated mRNA compositions produced by the methods described herein are more pure than mRNA compositions have enhanced stability and are thus more purified than dilute mRNA compositions. Whether a composition is more pure than a dilute composition may be determined by methods known in the art, including separating a composition to be purified into multiple equivalent samples, purifying each by a different method, and comparing the contents of the resulting purified composition. A first mRNA composition comprising a lower abundance or of degradation than a second mRNA composition is said to be “more pure” than the second mRNA composition.

Some aspects of the present disclosure relate to methods of preparing an mRNA formulation. In some embodiments, the method comprises diluting an overconcentrated mRNA composition to form a dilute mRNA composition. In some embodiments, the method comprises diluting an overconcentrated mRNA composition to form a dilute mRNA composition, and mixing the dilute mRNA composition with one or more carrier compounds to produce an mRNA formulation. In some embodiments, the method comprises diluting an overconcentrated mRNA composition to form a dilute mRNA composition, wherein the overconcentrated mRNA composition comprises a gel or viscous liquid comprising mRNA in a concentration of at least 10 g/L, and mixing the dilute mRNA composition with one or more carrier compounds to produce an mRNA formulation.

In some embodiments, the method comprises diluting an overconcentrated mRNA composition to form a dilute mRNA composition, wherein the overconcentrated mRNA composition comprises a gel or viscous liquid comprising mRNA in a concentration of at least g/L. In some embodiments, the overconcentrated mRNA composition comprises a gel comprising mRNA in a concentration of at least 10 g/L. In some embodiments, the overconcentrated mRNA composition comprises a viscous liquid comprising mRNA in a concentration of at least 10 g/L. In some embodiments, the overconcentrated mRNA composition comprises a gel or viscous liquid comprising mRNA in a concentration of at least 10 (e.g., at least 6, at least 8, at least 10, at least 12, at least 14, at least 16, at least 18, at least 20, at least 22, at least 24) g/L. In some embodiments, the overconcentrated mRNA composition comprises a gel or viscous liquid comprising mRNA in a concentration of at least 6 g/L, at least 8 g/L, at least 10 g/L, at least 12 g/L, at least 14 g/L, at least 16 g/L, at least 18 g/L, at least 20 g/L, at least 22 g/L, at least 24 g/L. In some embodiments, the overconcentrated mRNA composition comprises a gel or viscous liquid comprising mRNA in a concentration of at least 10 g/L. In some embodiments, the overconcentrated mRNA composition comprises a gel or viscous liquid comprising mRNA in a concentration of at least 20 g/L.

In some embodiments, the method comprises diluting an overconcentrated mRNA composition to form a dilute mRNA composition, wherein the overconcentrated mRNA composition is stored at refrigerated temperatures. In some embodiments, the overconcentrated mRNA composition is stored at refrigerated temperatures for at least 1 (e.g., at least 0.5, at least 1, at least 5, at least 7, at least 14, at least 30, at least 90, at least 180) day. In some embodiments, the overconcentrated mRNA composition is stored at refrigerated temperatures for at least 0.5 days, at least 1 day, at least 5 days, at least 7 days, at least 14 days, at least 30 days, at least 90 days, at least 180 days. In some embodiments, the overconcentrated mRNA composition is stored at refrigerated temperatures for at least 1 day. In some embodiments, the overconcentrated mRNA composition is stored at refrigerated temperatures for at least 7 days. In some embodiments, the overconcentrated mRNA composition is stored at refrigerated temperatures for at least 90 days. In some embodiments, the overconcentrated mRNA composition is stored at refrigerated temperatures for 1 to 90 days.

In some embodiments, the method comprises diluting an overconcentrated mRNA composition to form a dilute mRNA composition, wherein the overconcentrated mRNA composition is produced by an IVT reaction and a concentration step. In some embodiments, the IVT reaction is a quantitative IVT (qIVT) reaction. In some embodiments, a purification step is performed following the IVT reaction. In some embodiments, the purification step is tangential flow filtration (TFF). In some embodiments, the overconcentrated mRNA composition is a gel. In some embodiments, the overconcentrated mRNA composition is a viscous liquid. In some embodiments, the overconcentrated mRNA composition is diafiltered before the dilute mRNA composition is prepared. In some embodiments, the overconcentrated mRNA composition is subjected to a downstream processing step before mixing the dilute mRNA composition with one or more carrier compounds. In some embodiments, the overconcentrated mRNA composition is subjected to a downstream processing step before the dilute mRNA composition is prepared. In some embodiments, the downstream processing step is a cap reaction step, and/or a chromatography step. In some embodiments, the overconcentrated mRNA composition is produced and diluted. In some embodiments, the overconcentrated mRNA composition is produced and diluted and optionally the downstream processing step is performed in a continuous manufacturing process. In some embodiments, the overconcentrated mRNA composition is produced and diluted and optionally the downstream processing step is performed in a non-continuous manufacturing process.

In some embodiments, the method comprises diluting an overconcentrated mRNA composition to form a dilute mRNA composition. In some embodiments, the dilute mRNA composition has a concentration range of 3 g/L to 6 g/L. In some embodiments, the dilute mRNA composition has a concentration range of 3-6 (e.g., 1-6, 1-5, 1-4, 1-3, 1-2, 2-6, 2-5, 2-4, 2-3, 3-6, 3-5, 3-4, 4-6, 4-5, 5-6) g/L. In some embodiments, the corresponding mRNA composition in a concentration of about 1 g/L, 2 g/L, 3 g/L, 4 g/L, 5 g/L, 6 g/L. In some embodiments, the corresponding mRNA composition in a concentration of about 1 g/L. In some embodiments, the corresponding mRNA composition in a concentration of about 2 g/L. In some embodiments, the corresponding mRNA composition in a concentration of about 3 g/L. In some embodiments, the corresponding mRNA composition in a concentration of about 4 g/L. In some embodiments, the corresponding mRNA composition in a concentration of about 5 g/L. In some embodiments, the corresponding mRNA composition in a concentration of about 6 g/L. In some embodiments, the corresponding mRNA composition in a concentration of about 3-6 g/L.

In some embodiments, the method comprises diluting an overconcentrated mRNA composition to form a dilute mRNA composition, and mixing the dilute mRNA composition with one or more carrier compounds to produce an mRNA formulation. In some embodiments, the one or more carrier compounds is a lipid. In some embodiments, the lipid comprises a lipid nanoparticle (LNP).

### In Vitro Transcription (IVT)

Some aspects relate to mRNAs produced by “in vitro transcription” or IVT. IVT methods produce (e.g., synthesize) an RNA transcript (e.g., mRNA transcript) by contacting a DNA template (e.g., a first input DNA and a second input DNA) with an RNA polymerase (e.g., a T7 RNA polymerase, a T7 RNA polymerase variant, etc.) under conditions that result in the production of the RNA transcript. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, a wild-type T7 polymerase is used in an IVT reaction. In some embodiments, a modified or mutant T7 polymerase is used in an IVT reaction. In some embodiments, a T7 RNA polymerase variant comprises an amino acid sequences that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

The composition of the highly concentrate mRNA may further include buffers, salts and one or more IVT reaction components. Alternatively, the composition may be purified and free of one or more IVT reaction components or other material.

### Purification Steps

In some embodiments the highly concentrated mRNA preparation is prepared from an mRNA solution that has been subjected to some purification steps. In some embodiments, the highly concentrated mRNA preparation is first diluted and then subjected to further purification steps.

In some embodiments, the mRNA may be precipitated and then filtered to separate the solution containing impurities or subject to chromatography. Non-limiting examples of impurities that may be removed by filtration or chromatography include salts (e.g., salts of an mRNA production process (e.g., in vitro transcription) and/or salts used to precipitate mRNA), proteins (e.g., in vitro transcription enzymes, DNases, proteinases, RNase III), and DNA from the precipitated mRNA. In some embodiments, the step of filtering comprises adding the precipitated mRNA and supernatant to a filter.

In some embodiments, filtering mRNA is achieved by tangential flow filtration (TFF), which comprises contacting precipitated mRNA in an mRNA composition with a TFF membrane. In TFF, a mRNA composition flows over a filtration membrane (TFF membrane) comprising pores, with the pores of the membrane being oriented perpendicular to the direction of flow. Components of the mRNA composition flow through the pores, if able, while components that do not pass through the pores are retained in the mRNA composition. TFF thus removes smaller impurities, such as peptide fragments, DNA fragments, amino acids, and nucleotides from a mRNA composition, while larger molecules, such as full-length RNA transcripts, are retained in the mRNA composition. Additionally, RNA polymerases may produce double-stranded RNA transcripts during IVT, comprising an RNA: RNA hybrid of a full-length RNA transcript and another RNA with a complementary sequence. The second RNA that is hybridized to the full-length RNA transcript may be another full-length RNA, or a smaller RNA that hybridizes to only a portion of the full-length transcript. Like DNA fragments produced by DNase digestion of DNA templates, these small RNAs may also be removed during TFF, so that fewer dsRNA molecules are present in the filtered RNA composition.

The size of the pores of the TFF membrane affect which components are filtered (removed) from the mRNA composition and which are retained in the mRNA composition. Generally, TFF membranes are characterized in terms of a molecular weight cutoff, with components smaller than the molecular weight cutoff being removed from the mRNA composition during TFF, while components larger than the molecular weight cutoff being retained in the mRNA composition. In some embodiments, the tangential flow filtration comprises using a TFF membrane with a molecular weight cutoff of 500 kDa or less, 200 kDa or less, 150 kDa or less, 100 kDa or less, 50 kDa or less, 40 kDa or less, 30 kDa or less, 20 kDa or less, or lower. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less. In some embodiments, the tangential flow filtration comprises using a TFF membrane with a molecular weight cutoff of 50 kDa or less. In some embodiments, the tangential flow filtration comprises using a TFF membrane with a molecular weight cutoff of 40 kDa or less. In some embodiments, the tangential flow filtration comprises using a TFF membrane with a molecular weight cutoff of 30 kDa or less. In some embodiments, the tangential flow filtration comprises using a TFF membrane with a molecular weight cutoff of 20 kDa or less. In some embodiments, the tangential flow filtration comprises using a TFF membrane with a molecular weight cutoff between 20 kDa and 200 kDa. In some embodiments, the tangential flow filtration comprises using a TFF membrane with a molecular weight cutoff between 20 kDa and 150 kDa. Removal of liquid from an mRNA composition (e.g., by filtration) reduces the volume of the mRNA composition. Thus, the loss of volume through filtration outweighs the loss of mRNA. Other methods include, for instance, as oligo-dT or high-performance liquid chromatography.

In some embodiments the mRNA preparation is a diafiltered gel. Diafiltration is a method that uses ultrafiltration membranes to remove, replace, or lower the concentration of salts or solvents from a nucleic acid containing solution. The process selectively utilizes permeable membrane filters to separate the components of solutions and suspensions based on their molecular size. This method may be used to produce a highly concentrated mRNA composition that is diafiltered gel.

Following filtration, a washing solution may be added to the mRNA to further remove any residual proteins or DNAs from the mRNA. A washing solution refers to a solution in which the solubility of mRNA is minimal. Generally, a washing solution contains a salt that is capable of precipitating RNA, and/or an alcohol. After mRNA is precipitated, aspirating supernatant removes many dissolved impurities from the mRNA composition. However, the precipitated mRNA contains many bound salt cations, and any remaining liquid in the mRNA composition still contains residual impurities. Adding a washing solution dilutes these impurities, such that after the washing solution is removed, the total abundance of impurities of the mRNA composition is reduced. Removing impurities by repeated steps of washing precipitated mRNA and removing the washing solution has the additional advantage of being able to use different washing solutions in successive iterations, which may enhance the efficiency of impurity removal. For example, the first washing solution added to precipitated mRNA may be a washing solution in which the salts used to precipitate the mRNA are soluble, to reduce the salt concentration of the mRNA composition after precipitation. Next, precipitated mRNA may be washed with a second washing solution containing an enzyme (e.g., DNase, RNase III, or protease) to digest an impurity that may be present in the mRNA composition. This washing solution may contain one or more other components (e.g., an enzyme cofactor, such as magnesium ions) that promote enzyme activity, to enhance degradation of the impurity targeted by the enzyme. The next washing solution may then contain components useful for removing proteins (e.g., enzymes or host cell proteins) from a composition.

The precipitated mRNA may then be resuspended in a solvent with a low concentration of impurities, or an impurity-free solvent, to dissolve the precipitated mRNA and produce a purified mRNA preparation. Depending on whether the highly concentrated mRNA composition is prepared before or after the purification steps, the concentration of the mRNA solution may be adjusted to create a high concentration or dilute preparation. Resuspension and dissolving of precipitated mRNA typically occurs after the salts and/or alcohol used to precipitate the mRNA, and other impurities, are removed by one or more washing steps. Resuspending precipitated mRNA in a solution in which the mRNA is soluble, such as an aqueous buffer with a low salt concentration, results in the precipitated mRNA becoming dissolved in the resuspension solution.

### Downstream Processing Steps

Aspects of the present disclosure may provide additional steps after the IVT reaction is complete. These additional steps may be referred to as downstream processing steps. Thus, in some embodiments the highly concentrated mRNA preparation is prepared from an mRNA solution that has been subjected to downstream processing steps. In some embodiments, the highly concentrated mRNA preparation is diluted and then subjected to further downstream processing steps.

A downstream processing step is a process that alters the mRNA or the composition prior to formulation of the mRNA into a drug product. These steps include, in some embodiments, purification steps. An exemplary downstream processing step is a step involving capping of the mRNA. In some embodiments the highly concentrated mRNA preparation has not yet been capped and thus the mRNA in the preparation does not comprise a cap. In some embodiments, the downstream processing step may be selected from any one of the following: a diafiltration step, a cap reaction step, or a chromatography step.

In some embodiments, the composition is a process intermediate. The process intermediate may be packaged for storage at refrigerated or colder temperatures. In some embodiments the composition is in a container having a volume capacity of at least one liter (L). In some embodiments, the composition is in a container having a volume capacity of at least 1 (e.g., at least 0.5, at least 1, at least 2.5, at least 5, at least 10, at least 25, at least 50, at least 100) liter(s). In some embodiments, the composition is in a container having a volume capacity of at least 0.5 L, at least 1 L, at least 2.5 L, at least 5 L, at least 10 L, at least 25 L, at least 50 L, at least 100 L. In some embodiments, the composition is in a container having a volume capacity of at least 0.5 L. In some embodiments, the composition is in a container having a volume capacity of at least 1 L. In some embodiments, the composition is in a container having a volume capacity of at least 2.5 L. In some embodiments, the composition is in a container having a volume capacity of at least 5 L. In some embodiments, the composition is in a container having a volume capacity of at least 10 L. In some embodiments, the composition is in a container having a volume capacity of at least 25 L. In some embodiments, the composition is in a container having a volume capacity of at least 50 L. In some embodiments, the composition is in a container having a volume capacity of at least 100 L.

In some embodiments, these steps may be performed continuously or non-continuously with the IVT reaction. A continuous process is a process in which the highly concentrated mRNA is further manipulated until a drug product is formed. In contrast, a non-continuous process involves one or more storage steps. Thus, the highly concentrated mRNA may be stored prior to any further processing. In some embodiments the highly concentrated mRNA may be stored at refrigerated or frozen temperatures for an extended period of time. In some embodiments, the highly concentrated mRNA may be stored for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, 100, 150, 200, 250, 300, 350 or 400 days. In each instance, the highly concentrated mRNA may be stored for less than 500, 400, 300, 200, 150, 100, 90, 85, 80, 75, 70, 65, 60, 55, 50, 40, 35, 30, 25, or 20 days.

In some embodiments, the mRNA, may be further processed to produce a “dilute mRNA formulation”. In some embodiments, the dilute mRNA formulation may have a concentration ranging from 3-6 g/L. In some embodiments, the dilute mRNA formulation may have a concentration of 1-6 g/L, 2-6 g/L, 3-6 g/L, 4-6 g/L, 5-6 g/L. The dilute mRNA formulation may be used to further produce a drug product.

### Drug Product

Aspects of the present disclosure provide methods of preparing a mRNA formulation such as a pharmaceutical sample such as a drug product. In some embodiments, a drug product comprises a carrier, such as a charged carrier. Carriers include but are not limited to proteins and lipids. In some embodiments the carrier is a lipid nanoparticle (LNP) comprising an ionizable lipid, a structural lipid, a phospholipid, and a target mRNA. In some embodiments, the LNP comprises an ionizable lipid, a PEG-modified lipid, a phospholipid and a structural lipid.

In some aspects, the present disclosure provides a composition comprising mRNA formulated in a lipid nanoparticle, wherein the mRNA is prepared from a highly concentrated mRNA preparation prior to formulation in the lipid nanoparticle.

In some embodiments the mRNA drug product comprises a single mRNA formulation in an LNP. The methods disclosed herein may be used prepare the mRNA formulation and/or store the mRNA formulation.

Certain mRNA drug products can include multiple mRNAs and the methods disclosed herein can be used to prepare those different mRNAs. In some embodiments the mixtures comprise more than 1 mRNA of similar sizes. mRNAs have similar sizes if the mRNAs are within about 100 nucleotides of one another. mRNAs of different sizes have a size differential of greater than 100 nucleotides. In some embodiments the mixtures comprise about 2-50, 2-45, 2-40, 2-35, 2-30, 2-25, 2-20, 2-15, 2-14, 2-13, 2-12, 2-11, 2-10, 2-9, 2-8, 2-7, 2-6, 2-5, 2-4, 2-3, 3-50, 3-45, 3-40, 3-35, 3-30, 3-25, 3-20, 3-15, 3-14, 3-13, 3-12, 3-11, 3-10, 3-9, 3-8, 3-7, 3-6, 3-5, 3-4, 4-50, 4-45, 4-40, 4-35, 4-30, 4-25, 4-20, 4-15, 4-14, 4-13, 4-12, 4-11, 4-10, 4-9, 4-8, 4-7, 4-6, 4-5, 5-50, 5-45, 5-40, 5-35, 5-30, 5-25, 5-20, 5-15, 5-14, 5-13, 5-12, 5-11, 5-10, 5-9, 5-8, 5-7, 5-6, 2, 6-20, 6-15, 6-14, 6-13, 6-12, 6-11, 6-10, 6-9, 6-8, 6-7, 7-20, 7-15, 7-14, 7-13, 7-12, 7-11, 7-10, 7-9, 7-8, 8-20, 8-15, 8-14, 8-13, 8-12, 8-11, 8-10, 8-9, 9-20, 9-15, 9-14, 9-13, 9-12, 9-11, 9-10, 10-20, 10-15, 10-14, 10-13, 10-12, or 10-11 mRNA of similar sizes or different sizes.

The drug products may include populations of molecules. As used herein, a “population” of RNA molecules generally refers to a preparation comprising a plurality of copies of the molecule (e.g., mRNA) of interest. In some embodiments, a population is a homogenous population comprising a single mRNA species. As used herein, an mRNA species refers to an mRNA molecule having a given nucleotide sequence. Two or more mRNA molecules having identical nucleotide sequences and backbone compositions belong to the same mRNA species, while two mRNA molecules having different nucleotide sequences and/or different backbone compositions belong to different mRNA species. In some embodiments, a population a heterogenous population comprising two or more mRNA species. In some embodiments, a heterogenous population comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, or more mRNA species.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos.: WO2017075531, WO2015199952, WO2013086354, or WO2013116126, or selected from formulae CLI-CLXXXXII of U.S. Pat. No. 7,404,969; each of which is hereby incorporated by reference in its entirety for this purpose.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In some embodiments, the ionizable amino lipid may have a positively charged hydrophilic head and a hydrophobic tail that are connected via a linker structure. In addition to these, an ionizable lipid may also be a lipid including a cyclic amine group.

### Unique Codes

In some embodiments, mRNA drug products comprise a multivalent mRNA composition. In some embodiments multivalent RNA compositions may further comprise unique codes. In some embodiments, the mRNA unique codes are used to identify the presence of mRNA or determine a relative ratio of different mRNAs in a mixture (e.g., a reaction product or a drug product), using routine methods for identifying unique sequences. In some embodiments the unique codes may also serve as a template sequence for the nucleic acid sequence of the tags disclosed herein. In some embodiments the unique codes serve both functions.

Thus, aspects of the disclosure relate to methods of determining purity of mRNA compositions, wherein the target mRNA comprises one or more (e.g., 1, 2, 3, 4, or more) unique identifier sequences or unique code sequences. As used herein, an “identifier sequence” or “unique code sequence” refers to a sequence of a biological molecule (e.g., nucleic acid) that when combined with the sequence of another biological molecule serves to identify the other biological molecule. Typically, a unique code sequence is a heterologous sequence that is incorporated within or appended to a sequence of a target biological molecule and utilized as a reference in order to identify a target molecule of interest. In some embodiments, a unique code sequence is a sequence of a nucleic acid (e.g., a heterologous or synthetic nucleic acid) that is incorporated within or appended to a target nucleic acid and utilized as a reference in order to identify the target nucleic acid. In some embodiments, a unique code sequence is of the formula (N) n. In some embodiments, n is an integer in the range of 5 to 20, 5 to 10, 10 to 20, 7 to 20, or 7 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, N are each nucleotides that are independently selected from A, G, T, U, and C, or analogues thereof.

In some embodiments, one or more RNA species (e.g., RNA of a given sequence) of a RNA composition (e.g., a multivalent RNA composition) comprises a distinct unique codes. Unique codes may differ in sequence length, base composition, or sequence length and base composition. In some embodiments, each RNA species in a multivalent RNA composition comprises a unique code that differs from the unique code of every other mRNA in the multivalent RNA composition. In some embodiments, each RNA species in a multivalent RNA composition comprises a unique code with a different length. In some embodiments, each RNA species in a multivalent RNA composition comprises a unique code with length between 0 and 100, 0 and 50, 0 and 30, 0 and 20, 0 and 10, or 0 and 5 nucleotides. In some embodiments, each RNA species in a multivalent RNA composition comprises a unique code with length between 1 and 100, 1 and 50, 1 and 30, 1 and 20, 1 and 10, or 1 and 5 nucleotides.

In some embodiments, one or more in vitro transcribed mRNAs comprise one or more unique code sequences in an untranslated region (UTR), such as a 5′ UTR or 3′ UTR. Inclusion of a unique code sequence in the UTR of an mRNA prevents the unique code sequence from being translated into a peptide. In some embodiments, inclusion of a unique code in a UTR does not negatively affect the translation of (e.g., reduce translation of) the mRNA into a protein. In some embodiments, a unique code sequence is positioned in a 3′ UTR of an mRNA. In some embodiments, the unique code sequence is positioned upstream of the poly A tail of the mRNA. In some embodiments, the unique code sequence is positioned downstream of (e.g., after) the polyA tail of the mRNA. In some embodiments, the unique code sequence is positioned between the last codon of the ORF of the mRNA and the first “A” of the polyA tail of the mRNA. In some embodiments, a polynucleotide unique code positioned in a UTR comprises between 1 and 30 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides).

Exemplary unique code sequences include: GGAA, GGUUA, GACCA, GGACCA, GGCCAAA, GGCCAAGA, GGCCAAGGA, CCCGUACCCCC (SEQ ID NO: 12), AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; UGACCA; GACCUACGA; CCAA; GUUA; CCUUA; AGACC; UUACCA; GGAGGA; GUACGGA; GUUCAUU; GGCUUCUGACCA (SEQ ID NO: 1); GGCCACUCGUUAAGA (SEQ ID NO: 2); GGCCACUGAAGCCAUUGAAG (SEQ ID NO:3); GGCCACUGAAGCCAUUGUCAAGGA (SEQ ID NO: 4); GGCCACUGAAGCCAUUGUCACCGAA (SEQ ID NO: 5); GGCGAAGCACUCGUGGCCAUUCGCA (SEQ ID NO: 6); GGCCAAGGA; GGCCAAGGAA (SEQ ID NO: 7); GGCCAAGGAAA (SEQ ID NO: 8); GGCCACUGAAGA (SEQ ID NO: 9); GGCCACUGAAGCCAUU (SEQ ID NO: 10); or GGCCACUGAAGGAAG (SEQ ID NO: 11).

Nucleic Acids/mRNA Formulation and mRNA Process Intermediates

Aspects of the disclosure relate to methods of preparing mRNA formulation using high concentration mRNA process intermediates, which may comprise, in addition to the mRNA formulation, RNA fragments and other nucleic acids. As used herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid (e.g., mRNA) includes a 2′-O-alkylated ribose group. In some embodiments, a modified nucleic acid (e.g., mRNA) includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid (e.g., mRNA) is heterogeneous in backbone composition thereby containing any possible combination of polymer units linked together.

As described herein, a “mRNA” refers to messenger ribonucleic acid (mRNA), which is any ribonucleic acid (RNA) that encodes a (at least one) protein (a naturally occurring, non-naturally occurring, or modified polymer of amino acids) and can be translated to produce the encoded protein in vitro, in vivo, in situ, or ex vivo. In some embodiments, the mRNA may comprise one or more RNAs, each having an open reading frame (ORF). In some embodiments, each RNA (e.g., mRNA) further comprises a 5′ UTR, 3′ UTR, a poly(A) tail and/or a 5′ cap analog. It should also be understood that the mRNA of the present disclosure may include any 5′ untranslated region (UTR) and/or any 3′ UTR.

An open reading frame (ORF) is a continuous stretch of deoxyribonucleic acid (DNA) or RNA beginning with a start codon (e.g., methionine (ATG or AUG)) and ending with a stop codon (e.g., TAA, TAG or TGA, or UAA, UAG or UGA). An ORF typically encodes a protein. A protein may be an antigen such as a vaccine antigen or therapeutic or diagnostic protein in some embodiments. As used herein, a “vaccine antigen” is a biological preparation that improves immunity to a particular disease or infectious agent. Vaccine antigens encoded by an mRNA described herein may be utilized to treat conditions or diseases in many therapeutic areas such as, but not limited to, cancer, allergy and infectious disease. In some embodiments the cancer vaccines may be personalized cancer vaccines in the form of a concatemer or individual RNAs encoding peptide epitopes or a combination thereof.

A mRNA formulation is an mRNA present in a mixture which will be prepared. A mixture may include more than one mRNA. However, the mRNA that will be prepared according to the methods disclosed herein is a mRNA formulation. When the mixture includes more than one mRNA to be prepared, multiple mRNA formulations are present. A mRNA process intermediate is an mRNA present in a mixture at an intermediate step in the process of preparing an mRNA formulation or drug product.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved.

Modified nucleotides may include modified nucleobases. For example, a target mRNA provided herein may include a modified nucleobase selected from pseudouridine (v), 1-methylpseudouridine (mlv), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an mRNA includes a combination of at least two (e.g., 2, 3, 4 or more) of the foregoing modified nucleobases.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators). Such isolated molecules are those that are separated from their natural environment.

### Untranslated Regions

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the target mRNAs as flanking regions to an ORF. This publication is incorporated by reference herein for this purpose.

Additional exemplary UTRs that may be utilized in the nucleic acids provided herein include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

In some embodiments, the nucleic acid may comprise multiple UTRs, e.g., a double, a triple or a quadruple 5′ UTR or 3′ UTR.

### Poly(A) Tails

Some aspects relate to methods of preparing mRNA formulations containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP-IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP-IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP-IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm. Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (O-(2,3,4,5,6-Pentafluorobenzyl) hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl) phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Identification and Ratio Determination (IDR) Sequences

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### EXAMPLES

### Example 1: Preparation of Stable Overconcentrated mRNA Compositions

Highly concentrated (“overconcentrated”) mRNA compositions having enhanced stability have been prepared. The stability of these compositions has been analyzed using techniques for assessing the purity of the mRNA in the composition, for instance by tail purity and size purity. The tail purity provides a measure of the full-length mRNA in a mixture. A loss of full-length mRNA is indicative of mRNA instability or degradation. mRNA size purity is a measure of the overall sizes of nucleic acids in a mixture and provides an indication of mRNA instability or degradation. The highly concentrated mRNA compositions disclosed herein have enhanced stability relative to dilute mRNA formulations and can be stored and used effectively in downstream processing and formulation into drug product.

mRNA overconcentration events have occurred during the tangential flow filtration (TFF) unit operation used in the mRNA manufacturing process. The purpose of this experiment was to investigate and understand the effects of mRNA overconcentration on tail purity, a critical quality attribute. A quenched in-vitro transcription (qIVT) at 3.4 g/L mRNA was concentrated up to approximately 26.2 g/L without any buffer exchange, forming a viscous gel and high pressures within the TFF system. For reference, the target mRNA concentration for IVT TFF is 6 g/L. After diluting the overconcentrated mRNA to 6 g/L with water (WFI), and running samples on an HPLC system, there was no significant difference in % tail purity before and after the overconcentration event. Findings were confirmed using a paired t-test, which found that there is insufficient evidence to conclude that % tail purity is not equal before and after overconcentration. From this investigation, one can expect that an mRNA overconcentration event to gel formation during IVT TFF would not affect tail purity. Although only tail purity was evaluated in this investigation, it is worth noting that in a past investigation mRNA cap purity and size purity have been shown to not significantly change following an overconcentration to approximately 20 g/L during Final TFF.

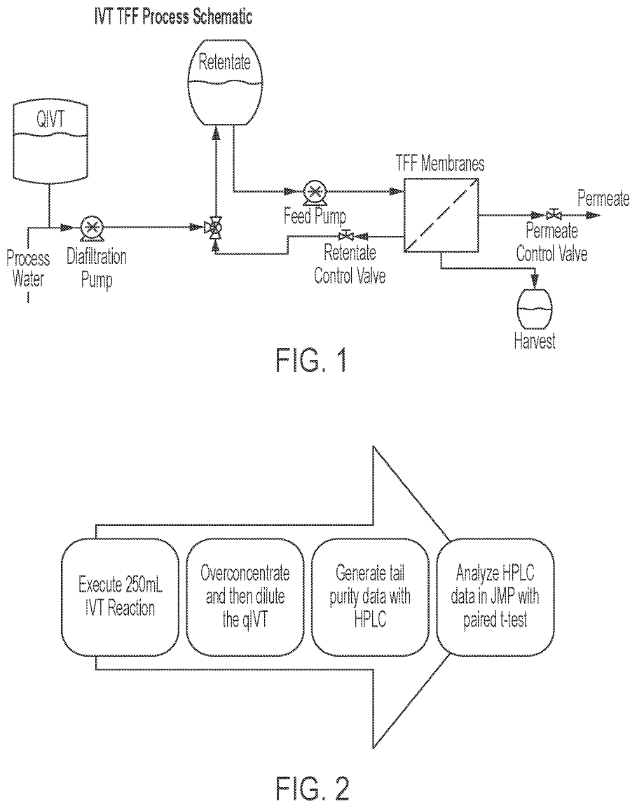

Tangential flow filtration (TFF) is a unit operation used in mRNA manufacturing to concentrate and buffer exchange mRNA process intermediates. mRNA overconcentration events have occurred during the IVT TFF unit operation used in the mRNA manufacturing process. The purpose of this study was to investigate and understand the effects of mRNA overconcentration on tail purity, a critical quality attribute. (FIG. 1).

A 250 mL in-vitro transcription (IVT) reaction was performed. Samples were collected from the quenched IVT (qIVT) before any TFF processing, which served as the control. The qIVT was then concentrated with TFF until gel formation occurred at approximately 26.2 g/L mRNA. The system could not process any further due over pressurization. The gel was extracted from the membrane using an air blow down, which relieved the high-pressure situation. This gelatinous feed volume was then diluted to its original target concentration of 6 g/L with water (WFI), and additional samples were collected from the qIVT. Tail purity was then measured by Nucleic Acid Process Development (NAPD) for all samples using reverse phase ion pair HPLC on a DNAPac column. Data was analyzed in JMP software using a paired t-test to evaluate if there was a significant difference in tail purity before and after the mRNA overconcentration event. (FIG. 2).

### Materials

### Raw Materials for IVT Reaction

The reactants needed to execute the IVT reaction are listed in Table 1.

TABLE 1
Raw Materials Used for 250 mL IVT Reaction

| | Raw Material | Order of | Concentration | Stock | Target | Volume | Volume |
|---|---|---|---|---|---|---|---|
| # | Input | Addition | Units | Concentration | Concentration | Added [mL] | to Add [mL] |
| 1 | 10X IVT Buffer | 1 | X | 10 | 1 | 25 | 25 |
| 2 | GTP, Tris | 2 | mM | 200 | 26.67 | 33.34 | 33.338 |
| 3 | ATP, Tris | 3 | mM | 200 | 13.33 | 16.66 | 16.663 |
| 4 | CTP, Tris | 4 | mM | 200 | 13.33 | 16.66 | 16.663 |
| 5 | N-1-Me-Pesudo- | 5 | mM | 200 | 6.67 | 8.34 | 8.338 |
| | UTP, Tris (G5) | | | | | | |
| 6 | Pyrophosphatase | 6 | U/mL | 100 | 1 | 2.5 | 2.5 |
| 7 | T7 WT RNA | 7 | U/mL | 50000 | 9000 | 45 | 45 |
| | Polymerase | | | | | | |
| 8 | WFI (Water) | 0 | | | | 93.75 | |
| 9 | TechDev-DNA- | 8 | mg/mL | 1 | 0.035 | 8.75 | 8.75 |
| | Circular-Plasmid | | | | | | |
| 10 | DNase | 9 | U/mL | 2000 | 50 | | 6.25 |
| | | | | | | | 25 |
| | | | | | SUM: | 250 | |

### TFF System

The Repligen KrosFlo KR2i Lab-Scale TFF System was used for this investigation.

### TFF Membrane

A Sartorious Sartocon Slice 200 Ultrafiltration Cassette was used during this investigation. The membrane material was Hydrosart, the Molecular Weight Cutoff (MWCO) was 30 kDa, and the effective filtration area was 0.02 m². The membrane was held in the Previous Slice Holder, with 20 Nm torque applied via an adjustable torque wrench.

### Results

The highly concentrated mRNA preparations were prepared by performing a 250 mL IVT reaction and concentrating the resulting mRNA to 26 mg/mL to form a gel. The mRNA was then diluted to a concentration of 6 mg/mL and diafiltered (water for injection (WFI) at 6 mg/mL). HPLC was performed on the dilute product to determine tail purity. A paired t-test was performed in JMP software to determine significance of the results.

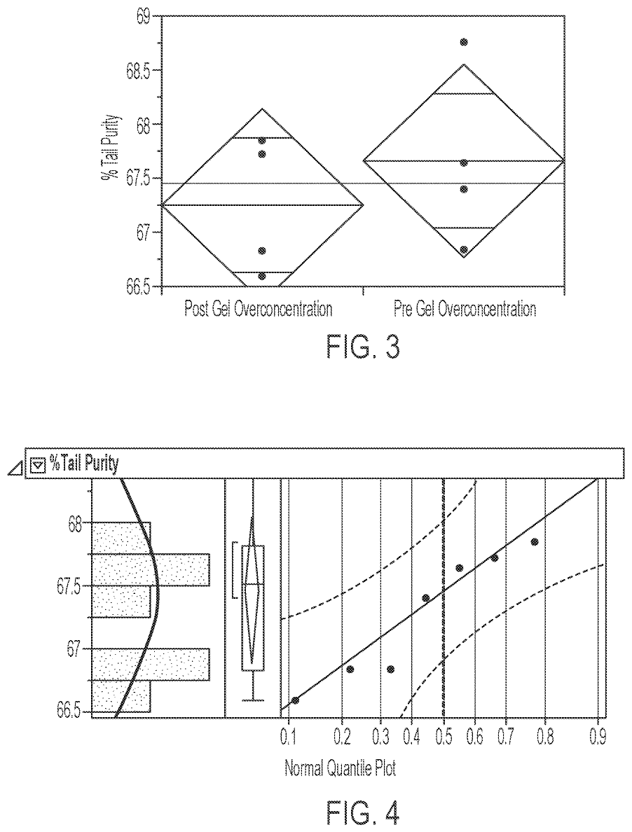

Estimated difference in tail purity is 0.41% before and after overconcentration. The P-value is 0.4547 (Table 2 and FIG. 3). Therefore, the population means are equal. In other words, tail purity is equal before and after concentration. See the statistical analysis appendix at the end of this memo for more detailed discussion.

TABLE 2
% Tail Purity Data

| | | HPLC Sample |
|---|---|---|
| | | Concentration |
| Sample # | % Tailed | (mg/mL) |
| Fresh qIVT Pre Gel Overconcentration, | 68.76 | 0.315 |
| Sample 1, Injection 1 | | |
| Fresh qIVT Pre Gel Overconcentration, | 67.40 | 0.320 |
| Sample 1, Injection 2 | | |
| Fresh qIVT Pre Gel Overconcentration, | 66.83 | 0.387 |
| Sample 2, Injection 1 | | |
| Fresh qIVT Pre Gel Overconcentration, | 67.64 | 0.369 |
| Sample 2, Injection 2 | | |
| Fresh qIVT Post Gel Overconcentration, | 67.72 | 0.259 |
| Sample 1, Injection 1 | | |
| Fresh qIVT Post Gel Overconcentration, | 66.83 | 0.266 |
| Sample 1, Injection 2 | | |
| Fresh qIVT Post Gel Overconcentration, | 66.59 | 0.274 |
| Sample 2, Injection 1 | | |
| Fresh qIVT Post Gel Overconcentration, | 67.85 | 0.266 |
| Sample 2, Injection 2 | | |

The results are shown in Table 3. The data demonstrate that a gel containing overconcentrated mRNA is stable. There was no significant degradation following the preparation of the overconcentrated material, as assessed by tail purity.

TABLE 3
Results of tail purity assay

| Sample | Average % Tail |
|---|---|
| Before gel overconcentration | 67.7% |
| After gel overconcentration | 67.3% |

There was no significant difference in tail purity before and after the IVT TFF mRNA overconcentration event studied. Findings were confirmed using a paired t-test, which found that tail purity is equal before and after overconcentration. Thus an mRNA overconcentration event leading to gel formation during IVT TFF would not significantly affect tail purity.

### Statistical Analysis Appendix

A paired t-test with two independent samples was used to analyze the HPLC data from this experiment. The null hypothesis (H0) is μD=0. The alternative hypothesis (H1) is that μD≠0.

Estimated difference in tail purity is 0.41% before and after overconcentration. The P-value is 0.4547 (FIG. 3).

The next step was to evaluate the two key assumptions of the t-test: (1) Does the data follow a normal distribution; and (2) Are variances approximately equal for the pre and post gel overconcentration data set?

The data, 8 samples in total, follows an approximately normal distribution since the normal quantile plot is approximately linear (FIG. 4). Normality is further supported by the fact that there is not enough evidence to reject the normality assumption with a Shapiro-Wilk or Anderson-Darling Goodness-of-Fit Test. The P value was approximately 0.53 in both tests, which is greater than 0.05.

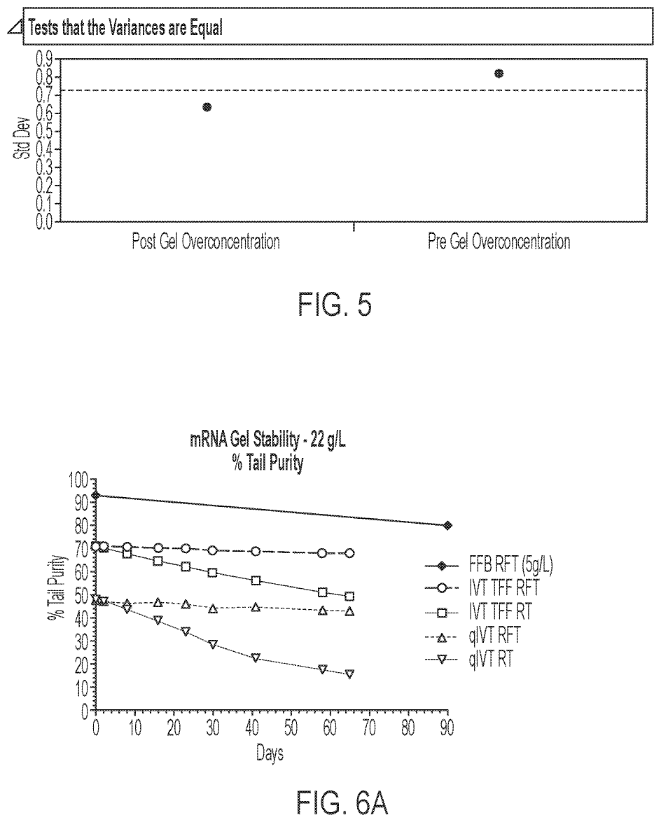

The second assumption of equal variance was also evaluated with t-tests. For a number of different tests, the p-value was far greater than 0.05 (FIG. 5). Therefore, the population means are equal. In other words, that variance is equal before and after concentration. Therefore, to conclude, the two assumptions of the performed paired t-test are both fair.

### Example 2: Analysis of Stable Overconcentrated mRNA Compositions

An experiment was conducted to analyze tail purity and size purity of highly concentrated mRNA preparations. Two types of highly concentrated mRNA preparations were investigated, a highly concentrated mRNA preparation made by a qIVT process and a highly concentrated mRNA preparation made by an IVT TFF process. Additionally, there were two storage temperatures used, 4° C. and room temperature (RT). The highly concentrated mRNA preparations were concentrated to a mRNA concentration of 22 g/L and compared to a dilute mRNA preparation of 5 g/L (FFB 5° C.), which is withing the range of usual concentration of mRNA preparations (i.e, 3-6 g/L). Timepoints were collected across 65 days. For the highly concentrated mRNA preparation, the 4° C. qIVT sample was a gel and the other three samples were viscous liquids.

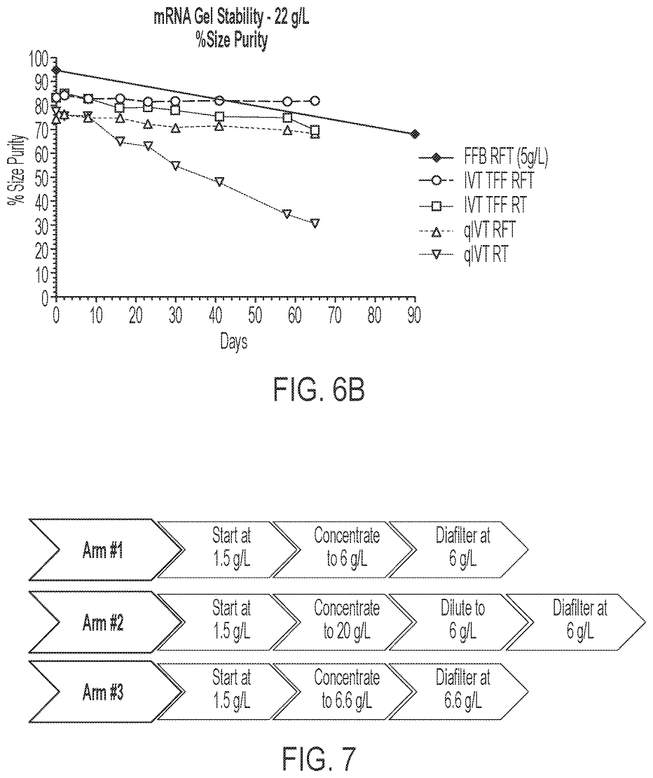

The results for tail purity of highly concentrated mRNA preparations (IVT TFF RT, IVT TFF 4° C., qIVT RT and qIVT 4° C.) relative to dilute mRNA preparations (FFB 5° C. 5 g/L), is reported in FIG. 6A and Table 4. It was found that the highly concentrated mRNA preparations stored at 4° C. was highly stable for at least 65 days and had better stability than dilute mRNA preparations stored at 5° C.

TABLE 4
Results of second tail purity assay

| | IVT | IVT | qIVT | qIVT | FFB |
|---|---|---|---|---|---|
| | TFF 4° C. | TFF RT | 4° C. | RT | 5° C. |
| | 22 g/L | 22 g/L | 22 g/L | 22 g/L | 5 g/L |
| Initial % Tail | 71.1% | 71.1% | 48.0% | 48.0% | 93.1% |
| Final % Tail | 68.0% | 49.4% | 43.1% | 15.3% | 80.2% |
| Days elapsed | 65 | 65 | 65 | 65 | 90 |
| Δ % Tail | 3.1% | 21.7% | 4.9% | 32.7% | 12.9% |
| Δ % Tail/Day | 0.05% | 0.33% | 0.08% | 0.50% | 0.14% |

The results for size purity of highly concentrated mRNA preparations (IVT TFF RT, IVT TFF 4° C., qIVT RT and qIVT 4° C.) relative to dilute mRNA preparations (FFB 5° C. 5 g/L), is reported in FIG. 6B and Table 5. It was found that the highly concentrated mRNA preparations stored at 4° C. was highly stable for at least 65 days and had better stability than dilute mRNA preparations stored at 5° C.

TABLE 5
Results of size purity assay

| | IVT | IVT | qIVT | qIVT | FFB |
|---|---|---|---|---|---|
| | TFF 4° C. | TFF RT | 4° C. | RT | 5° C. |
| | 22 g/L | 22 g/L | 22 g/L | 22 g/L | 5 g/L |
| Initial % Size | 83.5% | 83.5% | 76.3% | 76.3% | 95.0% |
| Final % Size | 82.2% | 70.1% | 68.6% | 30.9% | 68.0% |
| Days elapsed | 65 | 65 | 65 | 65 | 90 |
| Δ % Size | 1.3% | 13.4% | 7.7% | 45.4% | 27.0% |
| Δ % Size/Day | 0.02% | 0.21% | 0.12% | 0.70% | 0.30% |

Overall, these results demonstrate that highly concentrated mRNA preparations (e.g., IVT TFF or qIVT process intermediates) stored at 4° C. conditions were highly stable for at least 65 days. Additionally, highly concentrated mRNA preparations were more stable relative to dilute mRNA preparations when stored at 4° C. Specifically, for tail purity highly concentrated mRNA preparations were three times more stable for IVT TFF preparations and two times more stable for qIVT preparations. Finally, for size purity highly concentrated mRNA preparations were 15 times more stable for IVT TFF preparations and 2.5 times more stable for qIVT preparations.

### Example 3: MRNA Overconcentration and Freeze-Thaw does not Negatively Impact Product Quality Attributes

Final TFF is typically followed by a −20° C. freezing of the final filtered mRNA material, which may or may not be affected by the mRNA overconcentration event. The purpose of this experiment was to investigate and understand the effects of mRNA overconcentration and freeze-thaw on key product quality attributes, such as, % Tail Purity, % Size Purity, and % Cap Purity. Data generated from these three assays suggested that overconcentrating mRNA to 20 g/L had no degradation effect on any of the measured mRNA attributes. Additionally, a single freeze-thaw cycle had minimal degradation effects on any sample besides the highly concentrated sample (˜20 g/L). In other words, freezing mRNA at 6 g/L, regardless of whether it was previously overconcentrated, led to no significant degradation. This investigation suggests that mRNA intermediate material does not degrade following an overconcentration event during final TFF, and that overconcentrated mRNA can be safely frozen after dilution down to 6 g/L. From this investigation, one can expect that an mRNA overconcentration event to 20 g/L during Final TFF would not affect final product quality attributes. The process is depicted in FIG. 1.

### Experimental Design

This investigation consisted of two different experimental protocols (FIG. 7). Each experimental protocol started with the same dT2 Chromatography Elution sample before going through the different filtration conditions. This unfiltered starting dT2 chromatography Elution sample served as the control for all three experiments.

For each step of each experiment, two samples were aliquoted. Half of the samples were to be freeze-thawed, and the other half were simply kept at 4° C. No samples were frozen until all samples were gathered at the end of the third and final experiment. The freeze-thaw samples were frozen solid at −20° C. for two hours in a 2.0 mL microcentrifuge tube, then thawed for one hour at room temperature. No samples were diluted before freezing.

Experiment #1 represented a process strategy used in a 60 L IVT Scale mRNA manufacturing process. Following the elution from the preceding dT2 chromatography step, the process intermediate was at an mRNA concentration of about 1.5 g/L. This elution intermediate was concentrated up to 6 g/L, where it was then diafiltered into WFI.

Experiment #2 represented a realistic mRNA overconcentration event. Starting with the same 1.5 g/L dT2 chromatography elution, the mRNA was concentrated up to 20 g/L. The sample was then diluted back down to 6 g/L, where it was diafiltered into WFI.

At the end of each experiment, the sample was diafiltered for seven diavolumes into WFI. The samples were diafiltered into WFI instead of 32.5 mM sodium acetate. If no degradation effects were observed for the experimental conditions stored in WFI, normal stability behavior was expected in 32.5 mM sodium acetate as well.

### Materials

mRNA 1 dT2 Elution Process Intermediate Pool

mRNA 1 dT2 Elution Process Intermediate was pooled from previous Process Development investigations into an AKTA Avant Chromatography Scale Down Model. The dT2 elutions were stored at −20° C. for about five months before thawing for this investigation. Once thawed, the samples were pooled together into a Nalgene 2 L Biotainer, mixed gently, and then concentration was measured on a Nanodrop One UV/Vis Spectrophotometer. This mRNA 1 dT2 Elution Process Intermediate Pool served as the starting material and control for all three experimental arms. The total pool was around 800 mL, with a measured concentration of 1.51 mg/mL.

### TFF System

The Repligen KrosFlo KR2i Lab-Scale TFF System was used for this investigation.

### TFF Membrane

A Sartorious Sartocon Slice 200 Ultrafiltration Cassette was used during this investigation. The membrane material was Hydrosart, the Molecular Weight Cutoff (MWCO) was 30 kDa, and the effective filtration area was 0.02 m² (3081445902E--SW). The membrane was held in the Previous Slice Holder, with 20 Nm torque applied via an adjustable torque wrench.

### Methods

### TFF System Preparation

i) dT Neutralization Buffer was flushed through the TFF membrane to remove 0.1 N NaOH storage solution. Once the in-line conductivity sensor achieved a conductivity of 4.4 mS/cm, the feed pump was stopped.
ii) WFI was flushed through the TFF membrane to dT Neutralization Buffer. Once the in-line conductivity sensor achieved a conductivity of 0.0 mS/cm, the feed pump was stopped.
iii) Once the 0.0 mS/cm conductivity setpoint was achieved, the feed and retentate lines were lifted from the feed vessel to draw air. This purged all WFI from the feed and retentate lines, leaving just air in the system.
   (1) If leftover WFI is left in the system, it will dilute the mRNA feed sample, leading to inaccurate results.

### Concentration Step

i) An empty tube rack and conical tube were placed on the feed scale.
   (1) 250 ml conical tube was used for experiment #1.
   (2) 500 ml conical tube was used for experiment #2.
ii) The feed scale was tared.
iii) Using a serological pipette, the necessary volume of feed material was added to the empty conical tube on the feed scale.
   (1) Enough volume was added to achieve final concentration target.
      (a) ˜100 mL for experiment #1 (6.0 g/L)
      (b) ˜400 mL for experiment #2 (20 g/L)
      (c) ˜220 mL for similar version of for experiment #1 (6.6 g/L)
   (2) System hold-up volume in this case was ˜20 mL.
iv) The permeate line was clamped using a chemostat.
v) Process Operating Parameters were entered into the TFF System Machine-Human Interface.
   (1) 19 PSI TMP Setpoint
   (2) 100 mL/min pump flowrate (300 LMH Cross Flow Rate)
vi) The feed pump was operated in manual mode for about 5-10 minutes to let the system recirculate the mRNA sample.
   (1) While air is being pushed out of the retentate line, lift the retentate line exit out of the feed vessel to avoid bubbling up the feed.
   (2) Once air is purged, return retentate line to the feed vessel
vii) Once the TMP pressure setpoint is achieved, the system's pumps were stopped.
viii) The pump mode was changed from manual mode to C mode, and a calculated C factor was plugged in.
   (1) C Factor=Final Target mRNA Concentration/Initial mRNA Concentration
ix) The pump was started in C mode while the permeate line was still clamped with a chemostat.
x) Once the TMP setpoint was achieved, data collection was started on the TFF system software
   (1) 5 seconds per timepoint
xi) The chemostat on the permeate line was then removed, allowing the system to concentrate the feed material to the final target mRNA concentration.
xii) Once target concentration was achieved, the permeate line was again clamped with a chemostat. The pump was then operated in manual mode for 5-10 minutes.
   (1) Stop system data collection if it did not do so automatically
   (2) This method shears off mRNA possibly stuck to the membrane prior to the concentration reading
xiii) A 2.5 μL sample of the feed was aspirated from the feed vessel and measured on the Nanodrop One to get a measurement of mRNA concentration.
xiv) Two samples were aliquoted:
   (1) One sample was never frozen and kept at 4° C. in a 1 mL FluidX tube
   (2) The other sample was placed in a 2 mL microcentrifuge tube and kept at 4° C. until the sample freezing step (see 4.5).
   (3) Volumes of aliquots varied depending on mRNA concentration measurement.
      (a) Enough sample was added to have a total mass of 1.3 mg in approximately 1 mL of volume
   (4) No dilutions were performed for any of the samples to be frozen until after the samples thawed out.
      Dilution Step (Only Experiment 2 Required a Dilution Step to go from 20 g/L to 6 g/L Following its Concentration Step):
i) An mRNA concentration of 19.5 g/L was measured
ii) 56 mL of WFI was added to the ˜25 mL of mRNA sample, and the sample was vortexed for a few moments.
iii) The mRNA concentration was again measured to be exactly 6.00 g/L.

### Diafiltration Step

i) The feed scale and sample were already prepared during the preceding concentration step. The filtration unit was set up and feed and retentate lines were checked that they do not have air in them prior to starting. Once the TMP setpoint was achieved, data collection was started on the TFF system software

### Sample Freezing

All freeze-thaw samples were placed in a −20° C. freezer at the same start time. The samples sat in the freezer for about two hours and then thawed afterward at room temperature for about one hour. All samples froze solid in the two hours and thawed completely in the one hour.

### Sample Preparation for ATO

All samples were submitted to ATO-HT in 1 mL FluidX tubes. Each tube contained 1 mL of sample at a concentration of at least 1.3 mg/mL. Dilutions were performed using WFI. None of the freeze-thaw samples were diluted until after the freeze-thaw cycle.

### Purity Results

TABLE 6
% Purity Results

| Sample # | Arm | Checkpoint | Measured [mRNA] | % Tail | % Cap | % Size |
|---|---|---|---|---|---|---|
| 1 | Control | Starting Material | 1.51 | 76.2 | 100 | 59.5 |
| 2 | Control | Starting Material - F/T | 1.51 | 77 | 100 | 61.3 |
| 3 | 1 | Concentrated to 6 g/L | 6.00 | 78.8 | 100 | 64.1 |
| 4 | 1 | Concentrated to 6 g/L - F/T | 6.00 | 78 | 100 | 63 |
| 5 | 1 | Diafiltered at 6 g/L | 5.34 | 79.2 | 100 | 65.1 |
| 6 | 1 | Diafiltered at 6 g/L - F/T | 5.34 | 78.2 | 100 | 63.4 |
| 7 | 2 | Concentrated to 20 g/L | 19.50 | 79.3 | 100 | 64.8 |
| 8 | 2 | Concentrated to 20 g/L - F/T | 19.50 | 75.3 | 100 | 59 |
| 9 | 2 | Diluted Back to 6 g/L | 6.00 | 79.2 | 100 | 64.6 |
| 10 | 2 | Diluted Back to 6 g/L - F/T | 6.00 | 78 | 100 | 62.6 |
| 11 | 2 | Diafiltered at 6 g/L | 4.70 | 79.4 | 100 | 65.3 |
| 12 | 2 | Diafiltered at 6 g/L - F/T | 4.70 | 77.9 | 100 | 62.3 |
| 13 | 3 | Concentrated to 6.6 g/L | 6.60 | 79.1 | 100 | 64.4 |
| 14 | 3 | Concentrated to 6.6 g/L - F/T | 6.60 | 78.3 | 100 | 62.8 |
| 15 | 3 | Diafiltered at 6.6 g/L | 3.80 | 79.3 | 100 | 64.6 |
| 16 | 3 | Diafiltered at 6.6 g/L - F/T | 3.80 | 78.5 | 100 | 63.6 |

*Note:
“F/T” indicates that the sample went through a freeze-thaw cycle

These results suggest that concentrating mRNA up to 20 g/L during final TFF does not lead to detectable mRNA degradation. This is suggested by the fact that % Tail, % Cap, and % Size purity measurements all remained constant across the different arms and the control.

Furthermore, the single freeze-thaw cycle led to negligible mRNA degradation. The only significant degradation occurred between samples 7 and 8 (−4% Tail purity, −6% Size Purity). This was the only sample frozen at 20 g/L as opposed to 6 g/L, likely explaining the higher amount of degradation. The other freeze-thaw samples following the over concentration event, samples 10 and 12, had slightly higher but still low degradation after freezing. This suggests that overconcentration events may slightly accelerate mRNA degradation during freezing, but not significantly.

In conclusion, this data demonstrates that an mRNA overconcentration event to 20 g/L during Final TFF would not negatively affect final product quality attributes before and after freezing.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition comprising, a gel or viscous liquid comprising mRNA in a concentration of at least 10 g/L.

2. The composition of claim 1, wherein the composition is a process intermediate, packaged for storage at refrigerated or colder temperatures.

3. The composition of claim 2, wherein the composition is in a container having a volume capacity of at least one liter.

4. The composition of any one of claims 1-3, wherein the mRNA is in a concentration of at least 15 g/L.

5. The composition of any one of claims 1-3, wherein the mRNA is in a concentration of at least 20 g/L.

6. The composition of any one of claims 1-3, wherein the mRNA is in a concentration of at least 22 g/L.

7. The composition of any one of claims 1-3, wherein the mRNA is in a concentration of at least 25 g/L.

8. The composition of any one of claims 1-7, wherein the mRNA is in a concentration of less than 30-35 g/L.

9. The composition of any one of claims 1-8, wherein the mRNA has enhanced stability at refrigerated temperatures relative to a corresponding mRNA composition in a concentration of 3 g/L-6 g/L.

10. The composition of claim 9, wherein the enhanced stability is measured as tail purity and/or size purity.

11. The composition of any one of claims 1-10, wherein the mRNA does not comprise a cap.

12. The composition of any one of claims 1-10, wherein the composition comprises a diafiltered gel.

13. A method of preparing an mRNA formulation, comprising:
diluting an overconcentrated mRNA composition to form a dilute mRNA composition, wherein the overconcentrated mRNA composition comprises a gel or viscous liquid comprising mRNA in a concentration of at least 10 g/L, and
mixing the dilute mRNA composition with one or more carrier compounds to produce an mRNA formulation.

14. The method of claim 13, wherein the overconcentrated mRNA composition is stored at refrigerated temperatures.

15. The method of claim 13 or 14, wherein the overconcentrated mRNA composition is stored at refrigerated temperatures for at least 1 day.

16. The method of claim 13 or 14, wherein the overconcentrated mRNA composition is stored at refrigerated temperatures for 1 to 90 days.

17. The method of any one of claims 13-16, wherein the overconcentrated mRNA composition is concentrated to at least 20 g/L.

18. The method of any one of claims 13-16, wherein the overconcentrated mRNA composition is produced by an IVT reaction and a concentration step.

19. The method of claim 18, wherein the IVT reaction is a quantitative IVT (qIVT) reaction.

20. The method of claim 18, wherein a purification step is performed following the IVT reaction.

21. The method of claim 20, wherein the purification step is tangential flow filtration (TFF).

22. The method of any one of claims 13-21, wherein the overconcentrated mRNA composition is a gel.

23. The method of any one of claims 13-21, wherein the overconcentrated mRNA composition is a viscous liquid.

24. The method of any one of claims 13-23, wherein the overconcentrated mRNA composition is diafiltered before the dilute mRNA composition is prepared.

25. The method of any one of claims 13-24, wherein the overconcentrated mRNA composition is subjected to a downstream processing step before mixing the dilute mRNA composition with one or more carrier compounds.

26. The method of any one of claims 13-24, wherein the overconcentrated mRNA composition is subjected to a downstream processing step before the dilute mRNA composition is prepared.

27. The method of any one of claims 25-26, wherein the downstream processing step is a cap reaction step, and/or a chromatography step.

28. The method of any one of claims 18-27, wherein the overconcentrated mRNA composition is produced and diluted and optionally the downstream processing step is performed in a continuous manufacturing process.

29. The method of any one of claims 18-27, wherein the overconcentrated mRNA composition is produced and diluted and optionally the downstream processing step is performed in a in a non-continuous manufacturing process.

30. The method of any one of claims 13-29, wherein the dilute mRNA composition has a concentration range of 3 g/L to 6 g/L.

31. The method of any one of claims 13-30, wherein the one or more carrier compounds is a lipid.

32. The method of claim 31, wherein the lipid comprises a lipid nanoparticle (LNP).

33. The method of any one of claims 13-32, wherein the overconcentrated mRNA composition is a composition of any one of claims 1-12.


SEQUENCE LISTING (12 sequences)

SEQ ID NO: 1 - RNA, 12 nt, synthetic construct
  source 1..12: mol_type = other RNA
ggcttctgacca

SEQ ID NO: 2 - RNA, 15 nt, synthetic construct
  source 1..15: mol_type = other RNA
ggccactcgttaaga

SEQ ID NO: 3 - RNA, 20 nt, synthetic construct
  source 1..20: mol_type = other RNA
ggccactgaagccattgaag

SEQ ID NO: 4 - RNA, 24 nt, synthetic construct
  source 1..24: mol_type = other RNA
ggccactgaagccattgtcaagga

SEQ ID NO: 5 - RNA, 25 nt, synthetic construct
  source 1..25: mol_type = other RNA
ggccactgaagccattgtcaccgaa

SEQ ID NO: 6 - RNA, 25 nt, synthetic construct
  source 1..25: mol_type = other RNA
ggcgaagcactcgtggccattcgca

SEQ ID NO: 7 - RNA, 10 nt, synthetic construct
  source 1..10: mol_type = other RNA
ggccaaggaa

SEQ ID NO: 8 - RNA, 11 nt, synthetic construct
  source 1..11: mol_type = other RNA
ggccaaggaaa

SEQ ID NO: 9 - RNA, 12 nt, synthetic construct
  source 1..12: mol_type = other RNA
ggccactgaaga

SEQ ID NO: 10 - RNA, 16 nt, synthetic construct
  source 1..16: mol_type = other RNA
ggccactgaagccatt

SEQ ID NO: 11 - RNA, 15 nt, synthetic construct
  source 1..15: mol_type = other RNA
ggccactgaaggaag

SEQ ID NO: 12 - RNA, 11 nt, synthetic construct
  source 1..11: mol_type = other RNA
cccgtaccccc